(19) United States
(12) Patent Application Publication
(10) Pub. No.: US 2021/0268232 A1
(43) Pub. Date: Sep. 2, 2021

(54) CATHETER INSERTION SYSTEM, CATHETER INSERTION METHOD, AND INSERTION UNIT

(71) Applicant: TERUMO KABUSHIKI KAISHA, Tokyo (JP)

(72) Inventor: Hideki KATSURA, Ashigarakami-gun (JP)

(73) Assignee: TERUMO KABUSHIKI KAISHA, Tokyo (JP)

(21) Appl. No.: 17/325,351

(22) Filed: May 20, 2021

Related U.S. Application Data
(63) Continuation of application No. PCT/JP2019/030566, filed on Aug. 2, 2019.

(30) Foreign Application Priority Data
Nov. 20, 2018 (JP) 2018-217403

(51) Int. Cl. A61M 25/01; A61B 17/32

(52) U.S. Cl. CPC A61M 25/0113; A61B 2018/00982; A61B 17/320016

(57) ABSTRACT

A catheter insertion system enabling a catheter to be inserted smoothly, a catheter insertion method, and an insertion unit. A catheter insertion system includes: a first holding portion holding an elongated catheter and capable of moving forward in a axial direction of the catheter; a second holding portion holding the catheter on a side proximal as compared with the first holding portion and capable of moving forward in the axial direction of the catheter; and an insertion control unit controlling the operation of the first holding portion and the second holding portion. The insertion control unit controls the operation of the first holding portion and the second holding portion such that only the first holding portion moves forward in a state where the catheter is bent by the second holding portion being moved forward toward the first holding portion.

### CROSS-REFERENCES TO RELATED APPLICATIONS

This application is a continuation of International Application No. PCT/JP2019/030566 filed on Aug. 2, 2019, which claims priority to Japanese Patent Application No. 2018-217403 filed on Nov. 20, 2018, the entire content of both of which is incorporated herein by reference.

### FIELD OF THE DISCLOSURE

The present disclosure generally relates to a catheter insertion system, a catheter insertion method, and an insertion unit.

### BACKGROUND DISCUSSION

International Publication No. 2014/010207 discloses a robot arm that automatically inserts a catheter into a body lumen. The robot arm holds the proximal portion of the catheter and inserts the catheter into the body lumen by pushing the catheter into the body lumen.

In addition, another device may be connected to the proximal portion of the catheter. In this case, the catheter is pulled by the device connected to the proximal portion and is not smoothly inserted into the living body in the robot arm disclosed in International Publication No. 2014/010207.

### SUMMARY

A catheter insertion system is disclosed that enables a catheter to be inserted rather smoothly, a catheter insertion method, and an insertion unit.

A catheter insertion system is disclosed, the catheter insertion system includes: a first holding portion configured to hold an elongated catheter and configured to move forward in an axial direction of the catheter; a second holding portion configured to hold the catheter on a side proximal to the first holding portion and configured to move forward in the axial direction of the catheter; and an insertion control unit configured to control the operation of the first holding portion and the second holding portion, and wherein the insertion control unit is configured to control the operation of the first holding portion and the second holding portion such that only the first holding portion moves forward in a state where the catheter is bent by the second holding portion being moved forward toward the first holding portion.

A catheter insertion method is disclosed, which includes: holding an elongated catheter with a first holding portion and holding the catheter with a second holding portion, the second holding portion being on a side proximal of the first holding portion; bending the catheter by moving the second holding portion forward toward the first holding portion in an axial direction of the catheter; and inserting the catheter into a living body by moving only the first holding portion forward.

An insertion unit is disclosed for inserting an elongated catheter into a living body, the insertion unit comprising: a first holding portion configured to hold the elongated catheter and to move forward in an axial direction of the catheter; and a second holding portion configured to hold the catheter on a side proximal as compared with the first holding portion and configured to move forward along the axial direction of the catheter independently of the first holding portion.

According to the catheter insertion system and the catheter insertion method according to the disclosure, the catheter can be rather smoothly inserted into a living body.

### BRIEF DESCRIPTION OF THE DRAWINGS

FIG. 1 is a schematic view illustrating a catheter insertion system according to a first embodiment of the disclosure.

FIG. 2 is a block diagram of the catheter insertion system according to the first embodiment.

FIG. 3 is a schematic plan view illustrating a catheter of the catheter insertion system according to the first embodiment.

FIGS. 4A and 4B are enlarged partial cross-sectional views illustrating a distal portion of the catheter illustrated in FIG. 3.

FIG. 5 is a perspective view illustrating an insertion unit of the catheter insertion system according to the first embodiment.

FIGS. 6A-6D are diagrams for describing the operation of the insertion unit illustrated in FIG. 5.

FIG. 7 is a flowchart of a catheter insertion method according to the first embodiment.

FIG. 8 is a diagram illustrating an example in which the catheter insertion system according to the first embodiment is applied to a lung examination.

FIG. 9 is a block diagram of a catheter insertion system according to a second embodiment of the disclosure.

FIG. 10 is a schematic plan view illustrating a catheter of the catheter insertion system according to the second embodiment.

FIG. 11 is an enlarged partial cross-sectional view illustrating a distal portion of the catheter illustrated in FIG. 10.

FIGS. 12A-12D are diagrams for describing the operation of an insertion unit of the catheter insertion system according to the second embodiment.

FIGS. 13A-13C are diagrams illustrating an example in which the catheter insertion system according to the second embodiment is applied to a pancreatic examination.

### DETAILED DESCRIPTION

Set forth below with reference to the accompanying drawings is a detailed description of embodiments of a catheter insertion system, a catheter insertion method, and an insertion unit. Note that since embodiments described below are preferred specific examples of the present disclosure, although various technically preferable limitations are given, the scope of the present disclosure is not limited to the embodiments unless otherwise specified in the following descriptions. It should be noted that the dimensional ratios in the drawings are exaggerated for convenience of description and may differ from the actual ratios.

### First Embodiment

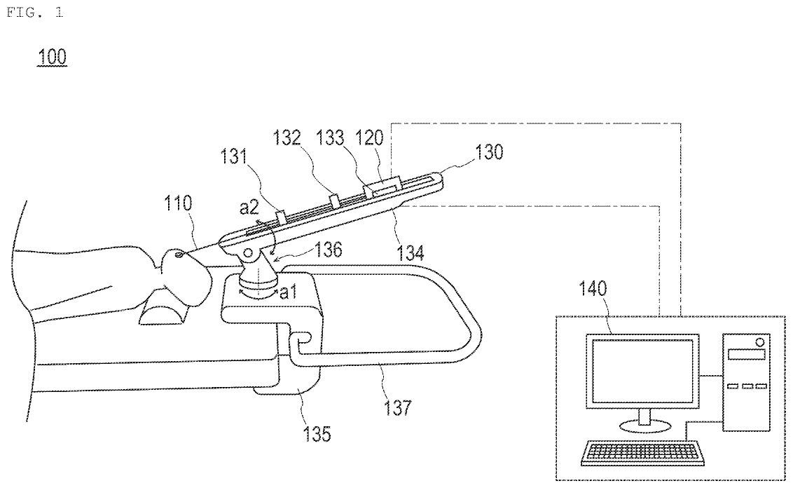

FIG. 1 is a schematic view illustrating a catheter insertion system 100 according to a first embodiment of the disclosure. FIG. 2 is a block diagram of the catheter insertion system 100 according to the first embodiment.

As illustrated in FIG. 1, the catheter insertion system 100 according to the present embodiment is a system inserting a catheter 110 into a body lumen in a state where a catheter operation unit 120 operating the operating elements of the catheter 110 is connected to the proximal portion of the catheter 110.

As illustrated in FIGS. 1 and 2, the catheter insertion system 100 according to the present embodiment includes the catheter 110, the catheter operation unit 120, an insertion unit 130, and a user terminal 140. Hereinafter, each part of the catheter insertion system 100 will be described in detail.

Catheter

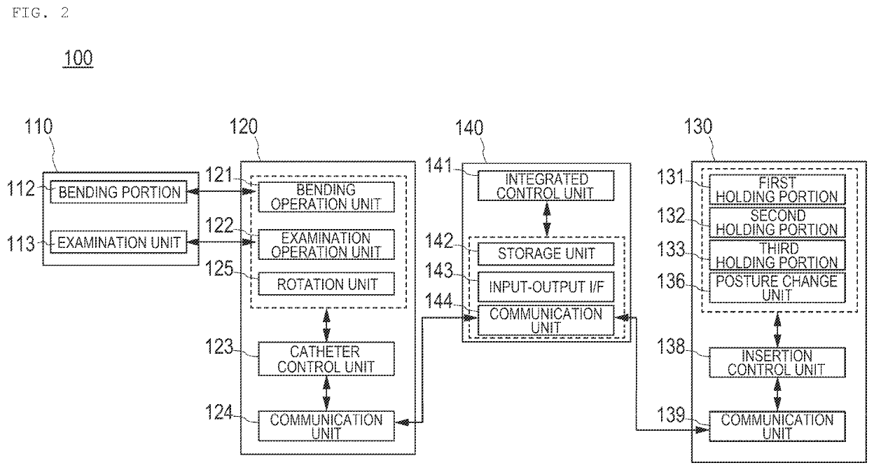

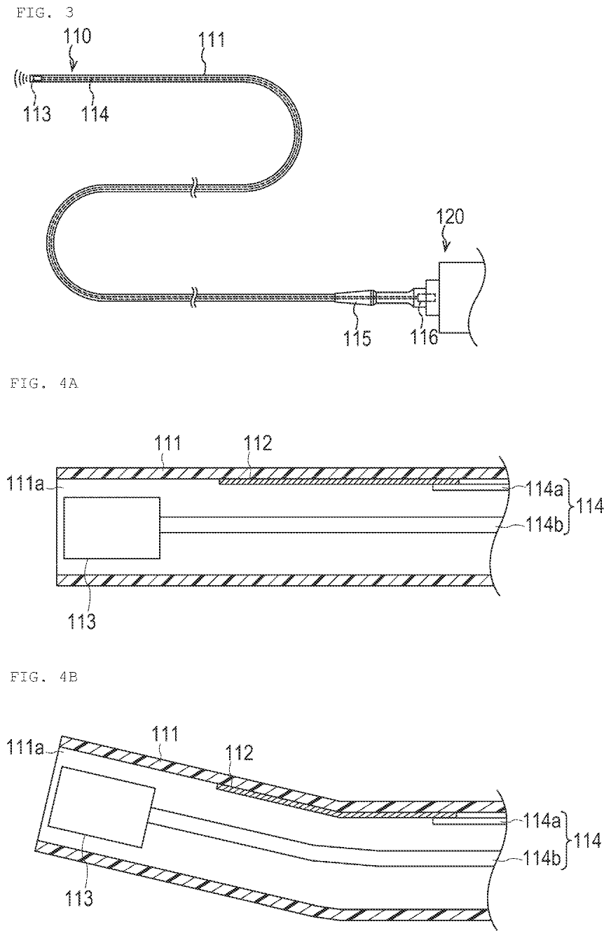

FIG. 3 is a schematic plan view illustrating the catheter 110 of the catheter insertion system 100 according to the first embodiment. FIGS. 4A and 4B are enlarged partial cross-sectional views illustrating the distal portion of the catheter 110 illustrated in FIG. 3.

As illustrated in FIGS. 3, 4A, and 4B, the catheter 110 according to the present embodiment includes an elongated sheath 111, a bending portion 112 where the distal portion of the sheath 111 can be bent, an examination unit 113 disposed in the distal portion of the sheath 111 for transmitting and receiving an examination wave, a signal line 114 connected to the bending portion 112 and the examination unit 113, and a hub 115 attached to the proximal portion of the sheath 111. The bending portion 112 and the examination unit 113 are the operating elements of the catheter 110. Hereinafter, each part of the catheter 110 will be described in detail.

It should be noted that the axial direction (longitudinal direction) of the catheter 110 will be referred to as “axial direction” in the following description. In the catheter 110, the side of insertion into a living body in the axial direction is referred to as “distal side” and the opposite side is referred to as “proximal side”. In addition, at each part of the catheter 110, the (most) distal end and a certain range from the distal end are referred to as “distal portion” and the (most) proximal end and a certain range from the proximal end are referred to as “proximal portion”.

The sheath 111 includes a lumen 111a where the examination unit 113 and the signal line 114 are disposed. The sheath 111 may be formed of a flexible material. The material of the sheath 111 is not particularly limited, and examples of the material of the sheath 111 can include various thermoplastic elastomers such as styrene-based, polyolefin-based, polyurethane-based, polyester-based, polyamide-based, polyimide-based, polybutadiene-based, transpolyisoprene-based, fluororubber-based, and chlorinated polyethylene-based and materials that can be combined with at least one of the elastomers (for example, polymer alloys, polymer blends, and laminates). In accordance with an exemplary embodiment, a hydrophilic lubricating coating layer exhibiting lubricity when wet can be disposed on the outer surface of the sheath 111.

The bending portion 112 can be, for example, an actuator that is bent as a result of a voltage application. Although such an actuator is not particularly limited, examples of the actuator can include those using an electroactive polymer (EAP). The bending portion 112 is disposed in the distal portion of the sheath 111 in the present embodiment. As illustrated in FIGS. 4A and 4B, the bending portion 112 is bent as a result of voltage application. Accordingly, the distal portion of the sheath 111 is bent as the bending portion 112 is deformed. However, the configuration of the bending portion 112 is not particularly limited insofar as the sheath 111 can be bent.

Although the examination unit 113 is not particularly limited insofar as it is capable of transmitting an examination wave in a living body and receiving the examination wave reflected by a biological tissue, the examination unit 113 can be, for example, an ultrasound transducer that transmits and receives an ultrasound wave and/or an optical element (lens or mirror) that transmits and receives light, for example, such as an infrared ray. The examination unit 113 transmits the examination wave toward the distal side in the axial direction of the sheath 111. It should be noted that the shape of the examination unit 113 is not particularly limited to the shape illustrated in FIGS. 3, 4A, and 4B although the examination unit 114 has a rectangular shape in FIGS. 3, 4A, and 4B.

The signal line 114 is connected to the bending portion 112 and the examination unit 113 and transmits a signal such as an electric signal and an optical signal. The signal line 114 has a first signal line 114a connected to the bending portion 112 and a second signal line 114b connected to the examination unit 113.

The hub 115 may have a cylindrical shape. As illustrated in FIG. 3, a connector unit 116 connected to the signal line 114 is disposed in the lumen of the hub 115. The material of the hub 115 is not particularly limited, and examples of the material of the hub 115 can include thermoplastic resins such as polycarbonate, polyamide, polysulfone, polyarylate, and a methacrylate-butylene-styrene copolymer.

In addition, a marker that has contrast under radioscopy or the like may be provided in the distal portion of the catheter 110.

Catheter Operation Unit

As illustrated in FIG. 2, the catheter operation unit 120 is connected to the proximal portion of the catheter 110 and operates the bending portion 112 and the examination unit 113, which are the operating elements of the catheter 110.

As illustrated in FIG. 2, the catheter operation unit 120 can include a bending operation unit 121, an examination operation unit 122, a catheter control unit 123, a communication unit 124, and a rotation unit 125. Hereinafter, each part of the catheter operation unit 120 will be described in detail.

The bending operation unit 121 generates an electric signal for operating (bending) the bending portion 112. The bending operation unit 121 can be a known voltage generator or the like. The bending operation unit 121 is electrically connected to the bending portion 112 via the first signal line 114a and the connector unit 116 (see FIGS. 3, 4A, and 4B) in a state where the catheter 110 and the catheter operation unit are connected to each other.

The examination operation unit 122 generates a signal such as an electric signal and an optical signal for operating the examination unit 113 (transmitting an examination wave) and receives a signal from the examination unit 113. The examination operation unit 122 is connected to the examination unit 113 via the second signal line 114b and the connector unit 116 (see FIGS. 3, 4A, and 4B) in a state where the catheter 110 and the catheter operation unit are connected to each other.

The catheter control unit 123 controls the operation of each part of the catheter operation unit 120 based on an instruction from an integrated control unit 141 of the user terminal 140, which will be described later. The catheter control unit 123 can includes a central processing unit (CPU), a random-access memory (RAM), a read-only memory (ROM), and the like. Predetermined operation control is performed by the CPU reading various programs pre-stored in the ROM into the RAM and executing the programs.

The communication unit 124 is an interface for communicating with the user terminal 140 and so on. It should be noted that the catheter operation unit 120 and the user terminal 140 are capable of adopting, for example, a wireless communication method using a communication function such as WiFi (registered trademark) and Bluetooth (registered trademark) or other non-contact wireless communication.

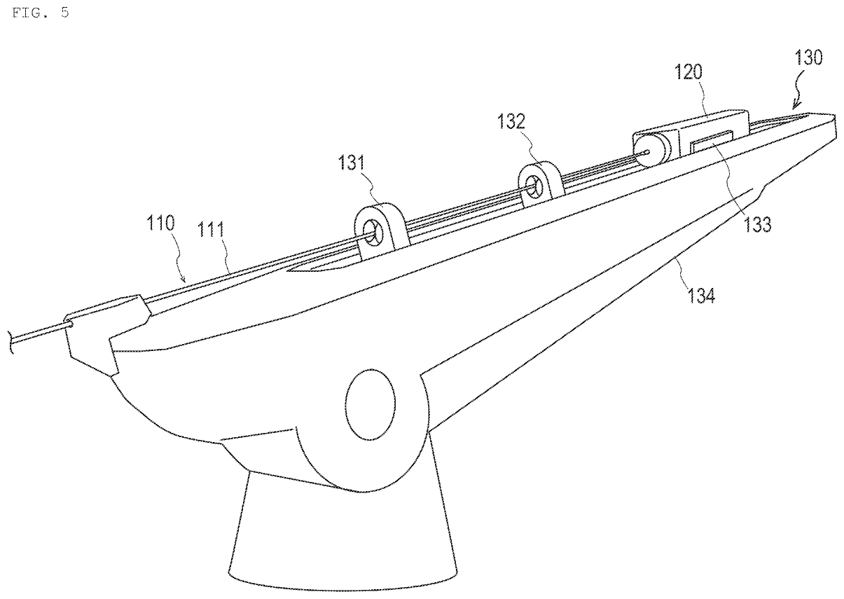

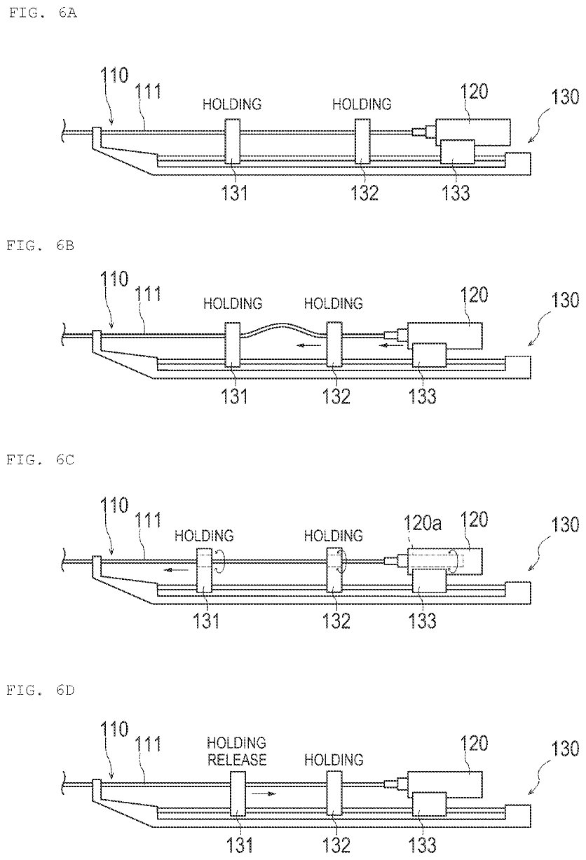

As illustrated in FIG. 6C, the rotation unit 125 rotates an internal component 120a (for example, the bending operation unit 121, the examination operation unit 122, the catheter control unit 123, the communication unit 124, and so on) of the catheter operation unit 120 so as to be interlocked with the rotation of a first holding portion 131 and a second holding portion 132 of the insertion unit 130, which will be described later.

Insertion Unit

FIG. 5 is a perspective view illustrating the insertion unit 130 of the catheter insertion system 100 according to the first embodiment. FIGS. 6A-6D are diagrams for describing the operation of the insertion unit 130 illustrated in FIG. 5. As illustrated in FIG. 5, the insertion unit 130 has the first holding portion 131, the second holding portion 132, a third holding portion 133, and a support 134. Hereinafter, each part of the insertion unit 130 will be described in detail.

The first holding portion 131 holds the sheath 111 of the catheter 110. The second holding portion 132 holds the sheath 111 of the catheter 110 on the side that is proximal as compared with the first holding portion 131. The third holding portion holds the catheter operation unit 120. In the present embodiment, each of the holding portions 131, 132, and 133 holds the object by sandwiching the object (i.e., the sheath 111 of the catheter 110 or the catheter operation unit 120). However, the method by which each of the holding portions 131, 132, and 133 holds the object is not particularly limited.

The first holding portion 131 is configured to be capable of switching between a state where the catheter 110 is held and a state where the catheter 110 is released.

Each of the holding portions 131, 132, and 133 includes a drive member such as a pusher and is capable of moving forward and backward along the axial direction of the catheter 110. It should be noted that “moving forward” in the present specification means moving to the distal side in the axial direction (in the direction of approaching the living body). In addition, “moving backward” in the present specification means moving to the proximal side in the axial direction (in the direction away from the living body).

The first holding portion 131 and the second holding portion 132 include a drive member such as a motor. As illustrated in FIG. 6C, the first holding portion 131 and the second holding portion 132 are configured to be capable of rotating the catheter 110 around a central axis of the catheter 110 in a state where the catheter 110 is held.

Next, the operation of each of the holding portions 131, 132, and 133 will be described. First, as illustrated in FIG. 6A, the first holding portion 131 holds the catheter 110, the second holding portion 132 holds the catheter 110 on the side that is proximal as compared with the first holding portion 131, and the third holding portion 133 holds the catheter operation unit 120. Next, the second holding portion 132 and the third holding portion 133 move forward toward the first holding portion 131 as illustrated in FIG. 6B. The catheter 110 bends as a result of the second holding portion 132 and the third holding portion 133 moving forward toward the first holding portion 131.

Next, only the first holding portion 131 moves forward as illustrated in FIG. 6C. As a result, the deflection of the catheter 110 is eliminated and the catheter 110 is inserted into the living body. It should be noted that each of the holding portions 131, 132, and 133 may rotate the catheter 110 and the internal component 120a of the catheter operation unit 120 around the central axis of the catheter 110 with the distal portion of the catheter 110 bent by the bending portion 112 at this time. As a result, the insertion unit 130 is capable of inserting the catheter 110 while adjusting the traveling direction of the catheter 110. Next, the first holding portion 131 releases the holding and moves backward as illustrated in FIG. 6D. Then, the insertion unit 130 is capable of gradually inserting the catheter 110 into the living body by repeating the operations illustrated in FIGS. 6A to 6D.

As illustrated in FIG. 1, the support 134 is configured to support the first holding portion 131, the second holding portion 132, and the third holding portion 133. The support 134 may be attached, for example, to a bed, an operating table, or the like by an attachment portion 135. In addition, the posture (i.e., orientation) of the support 134 may be adjusted by a posture change unit 136. The insertion angle of the catheter 110 can be adjusted as a result. In the present embodiment, the posture change unit 136 includes a drive member such as a servomotor and is capable of rotating the support 134 with respect to the attachment portion 135 as indicated by an arrow a1 in FIG. 1. In addition, in the present embodiment, the posture change unit 136 is capable of rotating the support 134 in the distal portion of the support 134 as indicated by an arrow a2 in FIG. 1. In addition, the attachment portion 135 may be provided with a barricade (or barrier) 137 in order to prevent a person from inadvertently coming into contact with the insertion unit 130.

As illustrated in FIG. 2, the insertion unit 130 further has an insertion control unit 138 and a communication unit 139.

The insertion control unit 138 controls the operation of each part of the insertion unit 130 based on an instruction from the integrated control unit 141 of the user terminal 140, which will be described later. The insertion control unit 138 can include a CPU, a RAM, a ROM, and the like. Predetermined operation control is performed by the CPU reading various programs pre-stored in the ROM into the RAM and executing the programs.

The communication unit 139 is an interface for communicating with the user terminal 140 and so on. It should be noted that the insertion unit 130 and the user terminal 140 are capable of adopting, for example, a wireless communication method using a communication function such as WiFi (registered trademark) and Bluetooth (registered trademark), other non-contact wireless communication, or wired communication.

User Terminal

The user terminal 140 has the integrated control unit 141, a storage unit 142, an input-output interface (I/F) 143, and a communication unit 144. The integrated control unit 141, the storage unit 142, the input-output I/F 143, and the communication unit 144 are connected to a bus and mutually exchange data or the like via the bus. Each part will be described below.

The integrated control unit 141 controls the operation of the catheter operation unit 120 and the insertion unit 130. In addition, the integrated control unit 141 controls the operation of each part of the user terminal 140. The integrated control unit 141 can include a CPU and, for example, controls each part and executes various types of arithmetic processing in accordance with various programs stored in the storage unit 142.

The storage unit 142 can include, for example, a ROM configured to store various programs and various data, a RAM configured to temporarily store a program and data as a work region, and a hard disk configured to store various data and various programs including an operating system (OS). The storage unit 142 may store, for example, a three-dimensional (3D) living body map acquired by computerized tomography (CT) or magnetic resonance imaging (MRI).

The input-output I/F 143 is an interface for connecting an input device such as a keyboard, a mouse, a scanner, and a microphone and an output device such as a display, a speaker, and a printer.

The communication unit 144 is an interface for communicating with the catheter operation unit 120, the insertion unit 130, and so on.

Catheter Insertion Method

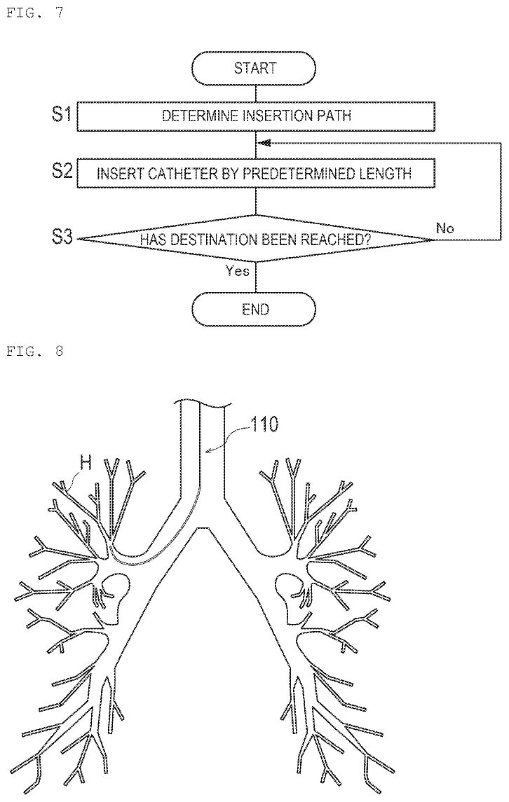

FIG. 7 is a flowchart of the catheter insertion method according to the first embodiment. FIG. 8 is a diagram illustrating an example in which the catheter insertion system 100 according to the first embodiment is applied to a lung examination. Hereinafter, the catheter insertion method according to the present embodiment will be described with reference to the example in which the catheter insertion system 100 according to the first embodiment is applied to the lung examination.

First, a user sets the catheter 110 and the catheter operation unit 120 in the insertion unit 130 as illustrated in FIG. 1. The lumen of the catheter 110 is filled with a priming solution (priming). The priming may be performed by the user or the catheter insertion system 100. In addition, the distal portion of the catheter 110, for example, is disposed in the mouth of the patient. The distal portion of the catheter 110 may be disposed by the user or the catheter insertion system 100 in the mouth of the patient.

Next, the user operates the user terminal 140 to instruct the catheter insertion system 100 to insert the catheter 110 into the living body. At this time, the user can set a catheter delivery destination or the like by operating the user terminal 140. Examples of the destination may include, for example, a bronchiole H in the vicinity of the alveoli as illustrated in FIG. 8. In accordance with an exemplary embodiment, the catheter 110, for example, may be smaller in outer diameter than an endoscope. Accordingly, the catheter 110 is capable of reaching the bronchiole H in the vicinity of the alveoli, which cannot be reached by the endoscope.

Next, as illustrated in FIG. 7, the integrated control unit 141 determines an insertion path based on a 3D living body map (Step S1).

Next, the integrated control unit 141 instructs the catheter control unit 123 and the insertion control unit 138 to insert the catheter 110 by a predetermined length along the insertion path (Step S2). Upon receiving the instruction, the catheter control unit 123 and the insertion control unit 138 insert the catheter 110 by the predetermined length along the insertion path.

In accordance with an exemplary embodiment, the catheter control unit 123 is instructed to transmit and receive an examination wave to and from the examination unit 113. As a result, the integrated control unit 141 is capable of grasping, for example, whether or not a biological tissue is present in the traveling direction of the catheter 110 and the distance to the biological tissue. It should be noted that the lungs lack blood or the like, unlike blood vessels, and thus light such as an infrared ray as well as an ultrasound wave can be used as the examination wave. Next, the integrated control unit 141 determines, for example, the traveling direction of the distal portion of the catheter 110 and the predetermined insertion length based on, for example, the reception signal of the examination wave and the insertion path determined in Step S1. Next, the integrated control unit 141 instructs the catheter control unit 123 and the insertion control unit 138 for the catheter 110 to be inserted by the predetermined length in the determined traveling direction. The catheter control unit 123 and the insertion control unit 138 control the operation of each part to insert the catheter 110 by the insertion length in the determined traveling direction. It should be noted that the operation of each of the holding portions 131, 132, and 133 at this time is as described above (see FIGS. 6A-6D) and thus will not be described here.

Next, the integrated control unit 141 determines whether or not the distal portion of the catheter 110 has reached the destination (Step S3). In accordance with an exemplary embodiment, for example, whether or not the distal portion of the catheter 110 has reached the destination can be determined based on, for example, the reception signal of the examination wave or the position of the marker provided in the distal portion of the catheter 110. The bronchi of the lungs have many branches and the like, and manual insertion of the catheter 110 requires proficiency. However, the catheter 110 can be automatically inserted by means of the catheter insertion system 100, and thus the operator does not have to be as proficient.

In a case where it is determined that the distal portion of the catheter 110 has reached the destination (Step S3: Yes), the insertion of the catheter 110 by the catheter insertion system 100 is completed. After the destination is reached by the catheter 110, the user or the catheter insertion system 100 performs a necessary examination or treatment. For example, the user or the catheter insertion system 100 performs intrapulmonary imaging or biological tissue collection from the lungs.

In a case where it is determined that the distal portion of the catheter 110 has not reached the destination (Step S3: No), the catheter insertion system 100 re-executes Step S2.

Although the catheter insertion method according to the present embodiment has been described above, the catheter insertion method is not limited to the above. For example, the body lumen into which the catheter 110 is inserted is not limited to the bronchi of the lungs and may be, for example, a blood vessel, a urethra, and so on. In addition, the catheter may be inserted during an examination wave-based examination, for example, although an example in which the catheter is inserted by a predetermined length after an examination wave-based examination has been described above. In addition, the insertion path may be changed during the insertion of the catheter 110.

Action and Effect

As described above, the catheter insertion system 100 according to the above embodiment has the first holding portion 131 holding the elongated catheter 110 and capable of moving forward in the axial direction of the catheter 110, the second holding portion 132 holding the catheter 110 on the side that is proximal as compared with the first holding portion 131 and capable of moving forward in the axial direction of the catheter 110, and the insertion control unit 138 controlling the operation of the first holding portion 131 and the second holding portion 132. The insertion control unit 138 controls the operation of the first holding portion 131 and the second holding portion 132 such that only the first holding portion 131 moves forward in a state where the catheter 110 is bent by the second holding portion 132 being moved forward toward the first holding portion 131.

In addition, by the catheter insertion method according to the above embodiment, the elongated catheter 110 is held by the first holding portion 131 and the second holding portion 132 holds the catheter 110 on the side that is proximal as compared with the first holding portion 131. Then, the catheter 110 is bent by the second holding portion 132 being moved forward toward the first holding portion 131 in the axial direction of the catheter 110. Then, the catheter 110 is inserted into the living body by only the first holding portion 131 being moved forward.

According to the catheter insertion system 100 and the catheter insertion method, the catheter 110 is inserted into the living body by only the first holding portion 131 being moved forward in a state where the catheter 110 is bent by the first holding portion 131 and the second holding portion 132. Accordingly, it is possible to suppress tension attributable to another instrument connected to the proximal portion of the catheter 110 from being applied to the catheter 110 when the catheter 110 is inserted. Accordingly, the catheter 110 can be rather smoothly inserted according to the catheter insertion system 100 and the catheter insertion method.

In addition, the catheter insertion system 100 further has the catheter 110 including the elongated sheath 111 and the operating elements and the catheter operation unit 120 connected to the proximal portion of the sheath 111 and operating the operating elements. Accordingly, tension attributable to the catheter operation unit 120 from being applied to the catheter 110 can be suppressed when the catheter 110 is inserted. Accordingly, the catheter 110 can be inserted into the living body relatively smoothly while exhibiting the functions of the operating elements of the catheter according to the catheter insertion system 100.

In addition, the operating elements include the bending portion 112 capable of bending the distal portion of the sheath 111. Accordingly, the catheter insertion system 100 is capable of adjusting the traveling direction of the sheath 111 by bending the distal portion of the sheath 111 by means of the bending portion 112. As a result, the catheter insertion system 100 is capable of rather smoothly inserting the catheter 110 into the living body.

In addition, the operating elements may include the examination unit 113 disposed in the distal portion of the sheath 111, transmitting an examination wave forward, and receiving the examination wave reflected in a biological tissue. Accordingly, the catheter insertion system 100 is capable of examining the biological tissue in front of the examination unit 113 by means of the examination unit 113 and inserting the catheter 110 into the living body based on the result of the examination. As a result, the catheter insertion system 100 is capable of smoothly inserting the catheter 110 into the living body.

### Second Embodiment

FIG. 9 is a block diagram of a catheter insertion system 200 according to a second embodiment of the disclosure. The catheter insertion system 200 according to the second embodiment is different from the catheter insertion system 100 according to the first embodiment in that a catheter 210 is inserted during biological tissue cutting and a tubular member 211 as a passage for a medical elongated body is disposed between biological tissues.

The catheter insertion system 200 according to the second embodiment can include the catheter 210, a catheter operation unit 220, an insertion unit 230, and the user terminal 140. Hereinafter, each part of the catheter insertion system 200 according to the second embodiment will be described in detail. It should be noted that configurations similar to those of the catheter insertion system 100 according to the first embodiment are denoted by the same reference numerals and description of the same reference numerals is omitted.

Catheter

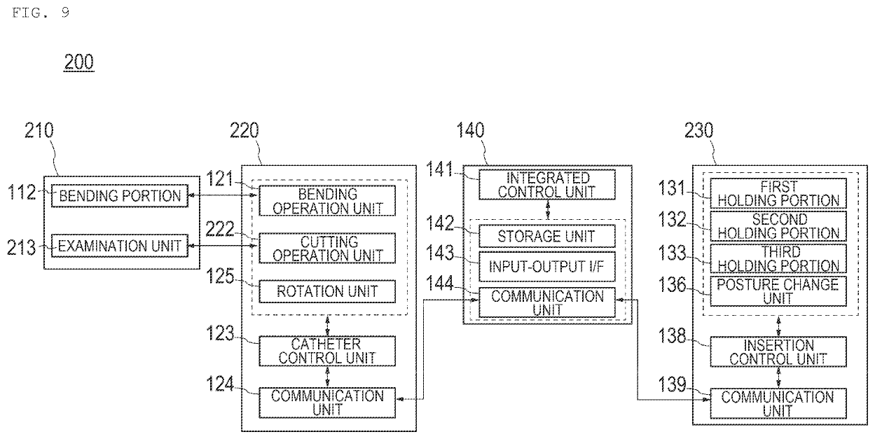

FIG. 10 is a schematic plan view illustrating the catheter 210 of the catheter insertion system 200 according to the second embodiment. FIG. 11 is an enlarged partial cross-sectional view illustrating the distal portion of the catheter 210 illustrated in FIG. 10. As illustrated in FIGS. 10 and 11, the catheter 210 has the sheath 111, the bending portion 112, a cutting unit 213, a signal line 214, the hub 115, and the tubular member 211. The bending portion 112 and the cutting unit 213 correspond to the operating elements of the catheter 210.

In accordance with an exemplary embodiment, the cutting unit 213 is disposed in the distal portion of the sheath 111 and cuts a biological tissue. The cutting unit 213, for example, can be a ball tip-type electric knife or the like although it is not particularly limited insofar as the cutting unit 213 is capable of cutting a biological tissue. It should be noted that the catheter according to the present embodiment may lack an examination unit and yet the catheter may have a bending portion, an examination unit, and a cutting unit without exception.

The signal line 214 has the first signal line 114a electrically connected to the bending portion 112 and a second signal line 214b electrically connected to the cutting unit 213.

As illustrated in FIG. 11, the tubular member 211 is disposed outside the sheath 111 and is turned back inside the sheath 111 at the distal end of the sheath 111. A turned-back part 211a of the tubular member 211 is extended to the outside of the sheath 111 by the sheath 111 being moved forward by the insertion unit 230 while the cutting unit, which will be described later, cuts a biological tissue. As a result, the tubular member 211 can be disposed between biological tissues. The tubular member 211 disposed between the biological tissues forms a passage for inserting another medical elongated body.

The tubular member 211 is not particularly limited insofar as the tubular member 211 is a biocompatible material. Examples of the biocompatible material of the tubular member 211 can include, for example, a biodegradable material such as a polymer selected from the group consisting of aliphatic polyester, polyester, polyacid anhydride, polyorthoester, polycarbonate, polyphosphazene, polyphosphate ester, polyvinyl alcohol, polypeptide, polysaccharide, protein, and cellulose.

In addition, a marker that has contrast under radioscopy may be provided in the distal portion of the catheter 210.

Catheter Operation Unit

As illustrated in FIG. 9, the catheter operation unit 220 can include the bending operation unit 121, a cutting operation unit 222, the catheter control unit 123, the communication unit 124, and the rotation unit 125.

Although the cutting operation unit 222 is not particularly limited, it can be configured by, for example, a current generator supplying a current to an electric knife.

Insertion Unit

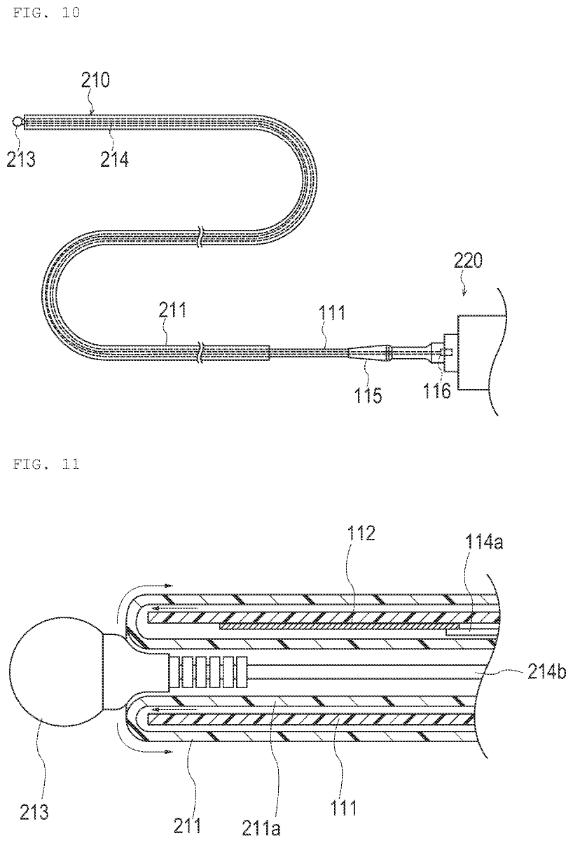

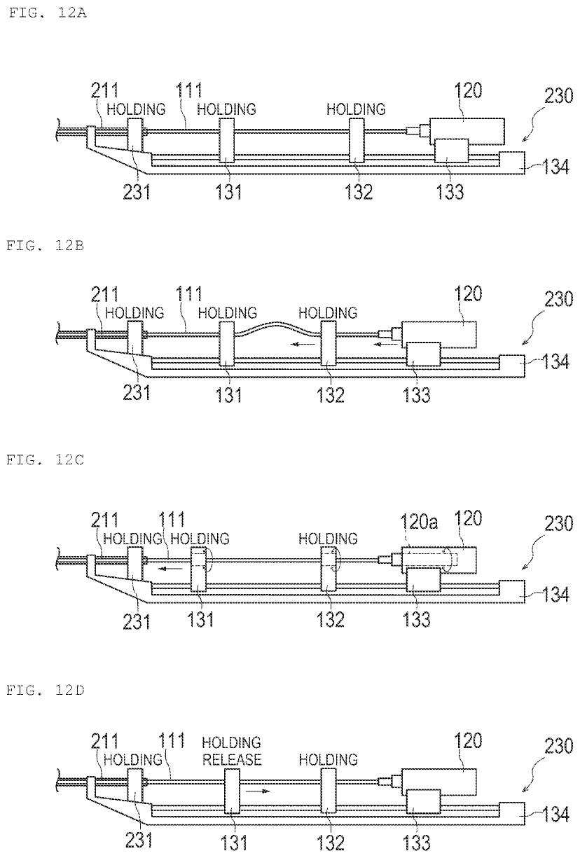

FIG. 12 is a diagram for describing the operation of the insertion unit 230 of the catheter insertion system 200 according to the second embodiment. As illustrated in FIG. 12, the insertion unit 230 can include the first holding portion 131, the second holding portion 132, the third holding portion 133, a fourth holding portion 231, and the support 134. In addition, the insertion unit 230 further has the insertion control unit 138 and the communication unit 139 as illustrated in FIG. 9.

As illustrated in FIGS. 12A-12D, the fourth holding portion 231 holds the proximal portion of the tubular member 211. The fourth holding portion 231 is fixed to the support 134.

Next, the operation of each of the holding portions 131, 132, 133, and 231 will be described. First, as illustrated in FIG. 12A, the first holding portion 131 holds the sheath 111, the second holding portion 132 holds the sheath 111 on the side that is proximal as compared with the first holding portion, the third holding portion holds the catheter operation unit 120, and the fourth holding portion 231 holds the proximal portion of the tubular member 211. Next, the second holding portion 132 and the third holding portion 133 move forward toward the first holding portion 131 as illustrated in FIG. 12B. The sheath 111 bends as a result of the second holding portion 132 and the third holding portion 133 moving forward toward the first holding portion 131. Next, as illustrated in FIG. 12C, only the first holding portion 131 moves forward with the cutting unit 213 operating. As a result, the sheath 111 is inserted into the living body. As the sheath 111 is inserted, the turned-back part 211a of the tubular member 211 is extended to the outside of the sheath 111 as illustrated in FIG. 11. Next, the first holding portion 131 releases the holding and moves backward as illustrated in FIG. 12D. Then, it is possible to dispose the tubular member 211 between the biological tissues by repeating the operations illustrated in FIGS. 12A to 12D.

Catheter Insertion Method

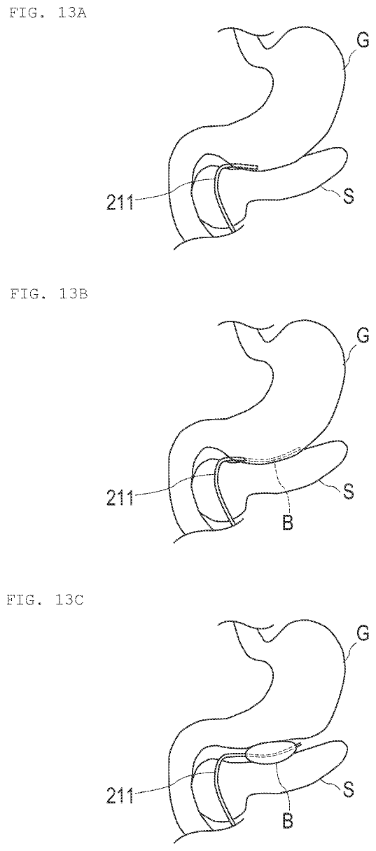

FIGS. 13A-13C are diagrams illustrating an example in which the catheter insertion system according to the second embodiment is applied to an examination of a pancreas S. Hereinafter, the catheter insertion method according to the present embodiment will be described with reference to the example in which the catheter insertion system 200 according to the second embodiment is applied to the examination of the pancreas S.

First, a user sets the catheter 210 and the catheter operation unit 220 in the insertion unit 230. The lumen of the catheter 210 is filled with a priming solution (priming). The priming may be performed by the user or the catheter insertion system 200. In addition, the distal portion of the catheter 210 is disposed, for example, on the body surface of the abdomen. The disposition of the distal portion of the catheter 210 may be performed by the user or the catheter insertion system 200.

Next, the user operates the user terminal 140 to instruct the catheter insertion system 200 to insert the catheter 110 into the living body. At this time, the user can set a catheter delivery destination by operating the user terminal 140. Examples of the destination include the part between a stomach G and the pancreas S as illustrated in FIG. 13A.

Next, the integrated control unit 141 determines an insertion path based on a 3D living body map (Step S1, see FIG. 7).

Next, the integrated control unit 141 instructs the catheter control unit 123 and the insertion control unit 138 to insert the catheter 110 by a predetermined length along the insertion path (Step S2, see FIG. 7). Upon receiving the instruction, the catheter control unit 123 and the insertion control unit 138 insert the catheter 210 by the predetermined length along the insertion path.

In accordance with an exemplary embodiment, the integrated control unit 141 first determines, for example, the traveling direction of the distal portion of the catheter 210 and the predetermined insertion length based on the insertion path. Next, the integrated control unit 141 instructs the catheter control unit 123 and the insertion control unit 138 for the catheter 210 to be inserted by the predetermined length in the determined traveling direction. The catheter control unit 123 and the insertion control unit 138 control the operation of each part to insert the catheter 210 by the insertion length in the determined traveling direction. It should be noted that the operation of each of the holding portions 131, 132, 133, and 231 at this time is as described above (see FIGS. 12A-12D) and thus will not be described here.

Next, the integrated control unit 141 determines whether or not the distal portion of the sheath 111 has reached the destination (Step S3). Whether or not the distal portion of the sheath 111 has reached the destination can be determined based on, for example, the position of the marker in the distal portion of the catheter 210.

In a case where it is determined, for example, that the distal portion of the sheath 111 has reached the destination (Step S3: Yes), the insertion of the catheter 210 by the catheter insertion system 200 is completed.

In accordance with an exemplary embodiment, an examination or treatment can be performed after the destination is reached by the catheter 210. The treatment may be performed by the user or the catheter insertion system 200. For example, as illustrated in FIGS. 13A and 13B, the sheath 111 and the cutting unit 213 may be removed from the tubular member 211 disposed between the biological tissues and a balloon catheter B may be inserted via the lumen of the tubular member 211. Next, the pancreas may be separated from the stomach by the balloon of the balloon catheter B being inflated as illustrated in FIG. 12C. In accordance with an exemplary embodiment, it is preferable to design the shape of the balloon by simulation or the like such that each organ performs a desired operation by the balloon being inflated. Next, the pancreas S may be imaged, for example, by a camera or a light source inserted into the lumen of the balloon catheter B.

In accordance with an exemplary embodiment, when it is determined that the distal portion of the sheath 111 has not reached the destination (Step S3: No), the catheter insertion system 100 performs Step S2 again.

Although the catheter insertion method according to the present embodiment has been described above, the catheter insertion method is not limited to the above. For example, the catheter delivery destination may not be the part between the pancreas and the stomach and may be parts between other organs or tissues, examples of which include those between the gallbladder and the pancreas and between the prostate and the bladder. In addition, after the tubular member 211 is disposed in the living body, for example, a medical instrument for biological tissue collection from the pancreas may be inserted without balloon catheter insertion into the lumen of the tubular member.

Action and Effect

In the catheter insertion system 200 according to the second embodiment, the operating elements include the cutting unit 213 disposed in the distal portion of the sheath 111 and cutting a biological tissue. Accordingly, the catheter insertion system 200 is capable of inserting the catheter 210 while cutting the biological tissue.

In addition, the catheter 210 further has the tubular member 211 disposed outside the sheath 111 and turned back inside the sheath 111 at the distal end of the sheath 111. The insertion control unit 138 controls the operation of the first holding portion 131 and the second holding portion 132 such that the sheath 111 is moved forward while the cutting unit 213 cuts the biological tissue and the turned-back part 211a of the tubular member 211 is extended to the outside of the sheath 111. Accordingly, the tubular member 211 can be disposed between biological tissues.

Although the catheter insertion system and the catheter insertion method according to the disclosure have been described above through the embodiments, the disclosure is not limited to the content described in the specification and can be changed as appropriate based on the description of the scope of claims.

For example, the catheter inserted by the catheter insertion system of the disclosure is not limited to the catheters according to the first embodiment and the second embodiment. For example, the catheter inserted by the catheter insertion system of the disclosure may be an ablation catheter provided with, for example, an electrode that cauterizes a biological tissue by a current flowing as an operating element. The catheter insertion system of the disclosure may be used for, for example, automatic ablation catheter insertion into a thin cardiac vein that is relatively complex in shape.

For example, although only the first holding portion is moved backward after the catheter is inserted by a predetermined length in the above embodiments (see FIGS. 6D and 12D), the first holding portion may not be moved backward after the catheter is inserted by the predetermined length. In addition, after the catheter is inserted by the predetermined length, the second holding portion as well as the first holding portion may be released and moved backward.

In addition, for example, the second holding portion and the third holding portion of the insertion unit may be integrally configured although the second holding portion and the third holding portion are configured as separate bodies in the above embodiments.

In addition, the means and methods for performing various types of processing in the catheter insertion system may be realized by either a dedicated hardware circuit or a programmed computer. In addition, the program may be provided online via a network such as the Internet.

The detailed description above describes embodiments of a catheter insertion system, a catheter insertion method, and an insertion unit. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A catheter insertion system comprising:
an elongated catheter;
a first holding portion configured to hold the catheter and configured to move forward in an axial direction of the catheter;
a second holding portion configured to hold the catheter on a side proximal to the first holding portion and configured to move forward in the axial direction of the catheter;
an insertion control unit configured to control the operation of the first holding portion and the second holding portion; and
wherein the insertion control unit is configured to control the operation of the first holding portion and the second holding portion such that only the first holding portion moves forward in a state where the catheter is bent by the second holding portion being moved forward toward the first holding portion.

2. The catheter insertion system according to claim 1, wherein
the catheter includes an elongated sheath and an operating element; and
a catheter operation unit connected to a proximal portion of the sheath and operating the operating element.

3. The catheter insertion system according to claim 2, wherein the operating element includes a bending portion capable of bending a distal portion of the sheath.

4. The catheter insertion system according to claim 2, wherein the operating element includes an examination unit disposed in the distal portion of the sheath, the examination unit configured to transmit an examination wave towards a biological tissue and to receive the examination wave reflected from the biological tissue.

5. The catheter insertion system according to claim 2, wherein the operating element includes a cutting unit disposed in the distal portion of the sheath and configured to cut a biological tissue.

6. The catheter insertion system according to claim 5, wherein
the catheter further includes a tubular member disposed outside of the sheath and turned back inside the sheath in the distal portion of the sheath; and
the insertion control unit is configured to control the operation of the first holding portion and the second holding portion such that the sheath is moved forward while the cutting unit cuts the biological tissue and the turned-back part of the tubular member is extended to the outside of the sheath.

7. The catheter insertion system according to claim 1, wherein each of the first holding portion and the second holding portion are configured to be capable of rotating the catheter around a central axis of the catheter.

8. The catheter insertion system according to claim 1, further comprising:
a support configured to support the first holding portion, the second holding portion, and the insertion control unit; and
the support including a posture change unit configured to adjust an orientation of the first holding portion, the second holding portion, and the insertion control unit relative to a living body during insertion of the catheter into the living body.

9. The catheter insertion system according to claim 8, further comprising:
a third holding portion arranged proximally to the second holding portion, the third holding portion configured to hold the insertion control unit; and
each of the first holding portion, the second holding portion, and the third holding portion is configured to be capable of switching between a state in which the catheter is held and a state in which the catheter is not held, and wherein the each of the first holding portion, the second holding portion, and the third holding portion are configured to move in the axial direction of the catheter on the support.

10. The catheter insertion system according to claim 9, wherein the catheter further includes a tubular member, the tubular member configured to be a passage for a medical elongated body; and
a fourth holding portion configured to hold a proximal portion of the tubular member, the fourth holding portion arranged proximally of the first holding portion, the second holding portion, and the third holding portion.

11. A catheter insertion method comprising:
holding an elongated catheter with a first holding portion and holding the catheter with a second holding portion, the second holding portion being on a side proximal to the first holding portion;
bending the catheter by moving the second holding portion forward toward the first holding portion in an axial direction of the catheter; and
inserting the catheter into a living body by moving only the first holding portion forward.

12. The catheter insertion method according to claim 11, further comprising:
gradually inserting the catheter into the living body by repeating the bending of the catheter by moving the second holding portion forward toward the first holding portion in the axial direction of the catheter and the moving only of the first holding portion forward.

13. The catheter insertion method according to claim 11, further comprising:
supporting the first holding portion, the second holding portion, and the insertion control unit on a support; and
adjusting an insertion angle of the catheter into the living body by adjusting an orientation angle of the support.

14. The catheter insertion method according to claim 11,
setting a catheter delivery destination for the catheter;
determining an insertion path for the catheter; and
inserting the catheter into the living body along the insertion path to the catheter delivery destination.

15. The catheter insertion method according to claim 11, further comprising:
transmitting an examination wave forward in a biological tissue of the living body from an examination unit disposed in a distal portion of the catheter and receiving the examination wave reflected in the biological tissue of the living body; and
using the received examination wave reflected in the biological tissue of the living body to guide the catheter to a catheter delivery destination.

16. The catheter insertion method according to claim 11, further comprising:
cutting a biological tissue in the living body with a cutting unit disposed in a distal portion of the catheter.

17. An insertion unit for inserting an elongated catheter into a living body, the insertion unit comprising:
a first holding portion configured to hold the elongated catheter and to move forward in an axial direction of the catheter; and
a second holding portion configured to hold the catheter on a side proximal as compared with the first holding portion and configured to move forward along the axial direction of the catheter independently of the first holding portion.

18. The insertion unit according to claim 17, wherein each of the first holding portion and the second holding portion are configured to be capable of rotating the catheter around a central axis of the catheter.

19. The insertion unit according to claim 17, further comprising:
a support configured to support the first holding portion, the second holding portion, and the insertion control unit; and
wherein the support includes a posture change unit configured to adjust an orientation of the first holding portion, the second holding portion, and the insertion control unit relative to the living body.

20. The insertion unit according to claim 19, further comprising:
a third holding portion arranged proximally to the second holding portion, the third holding portion configured to hold the insertion control unit; and
each of the first holding portion, the second holding portion, and the third holding portion is configured to be capable of switching between a state in which the catheter is held and a state in which the catheter is not held, and wherein the each of the first holding portion, the second holding portion, and the third holding portion are configured to move forward and backward in the axial direction of the catheter in the support.
